(19) United States
(12) Patent Application Publication
(10) Pub. No.: US 2003/0014024 A1
(43) Pub. Date: Jan. 16, 2003

(54) Reusable diaper with liner

(76) Inventor: Kamila Kiecker, Berwyn, IL (US)

Correspondence Address: Kamila Kiecker, 6945 W. 34th Street, Berwyn, IL, 60402, US

(21) Appl. No.: 09/902,814

(22) Filed: Jul. 12, 2001

(51) Int. Cl. A61F 13/15

(52) U.S. Cl. 604/358

(57) ABSTRACT

This is what is new in the invention, REUSABLE DIAPER WITH LINER; the nylon mesh liner; the structure of material around the waist of the diaper; the structure and attachment of the flannel fabric; the structure of the outer nylon shell; and the structure of the fastening flaps.

### CROSS-REFERENCE TO RELATED APPLICATIONS

Design Patent No. U.S. D436,400 S has been issued Jan. 16, 2001

REUSABLE DIAPER WITH LINER—Kiecker

### BACKGROUND OF THE INVENTION

Within the diaper industry, let it be know that I, KAMILA KIECKER, has researched and used the various existing reusable diapers, and I do not believe that my invention has been known or used in the United States before my invention thereof.

The following related All-In-One diapers are:

Kushies (U.S. Pat. No. 5,019,068)

Bumkins (www.bumkins.com—not patented).

The problems I have found in these products are that they leaked, took too long to dry, and were not comfortable enough around baby's legs and abdomen. I added the nylon mesh for easy clean-up, doubled the nylon outer shell, separated the layers of cloth liner for quick drying, and made it comfortable for baby's legs and abdomen.

### BRIEF SUMMARY OF THE INVENTION

The following invention REUSABLE DIAPER WITH LINER is an All-In-One diaper making it possible for easy and enjoyable diapering for mother. It promotes healthier body functions and creates respect for the environment.

### BRIEF DESCRIPTION OF THE SEVERAL VIEWS OF THE DRAWINGS

Be it known that I, KAMILA KIECKER, have invented a new, original, and structural design for a REUSABLE DIAPER WITH LINER, called WEE WEE DIAPER of which the following is a specification, reference being had to the accompanying drawing, forming a part hereof.

FIG. 1 is a front elevational view of the REUSABLE DIAPER of the present invention;

FIG. 2 is a rear plan view of the REUSABLE DIAPER of FIG. 1;

FIG. 3 is a top plan view of the REUSABLE DIAPER of FIG. 1 in an alternate position;

FIG. 4 is a right plan view of the REUSABLE DIAPER of FIG. 1, the left side elevation view being a mirror image thereof;

FIG. 5 is a bottom plan of the REUSABLE DIAPER of FIG. 1 in an alternate position;

FIG. 6 is a top plan view of the cloth liner for boys of the REUSABLE DIAPER of FIG. 1;

FIG. 7 is a bottom plan view of the cloth liner for boys of the REUSABLE DIAPER of FIG. 1;

FIG. 8 is a left side elevational view of the cloth liner for boys of the REUSABLE DIAPER of FIG. 1, the right side elevational view being a mirror image thereof;

FIG. 9 is a top plan view of the cloth liner for girls of the REUSABLE DIAPER of FIG. 1;

FIG. 10 is a bottom plan view of the cloth liner for girls of the REUSABLE DIAPER of FIG. 1;

FIG. 11 is a left side elevational view of the cloth liner for girls of the REUSABLE DIAPER of FIG. 1, the right side elevational view being a mirror image thereof.

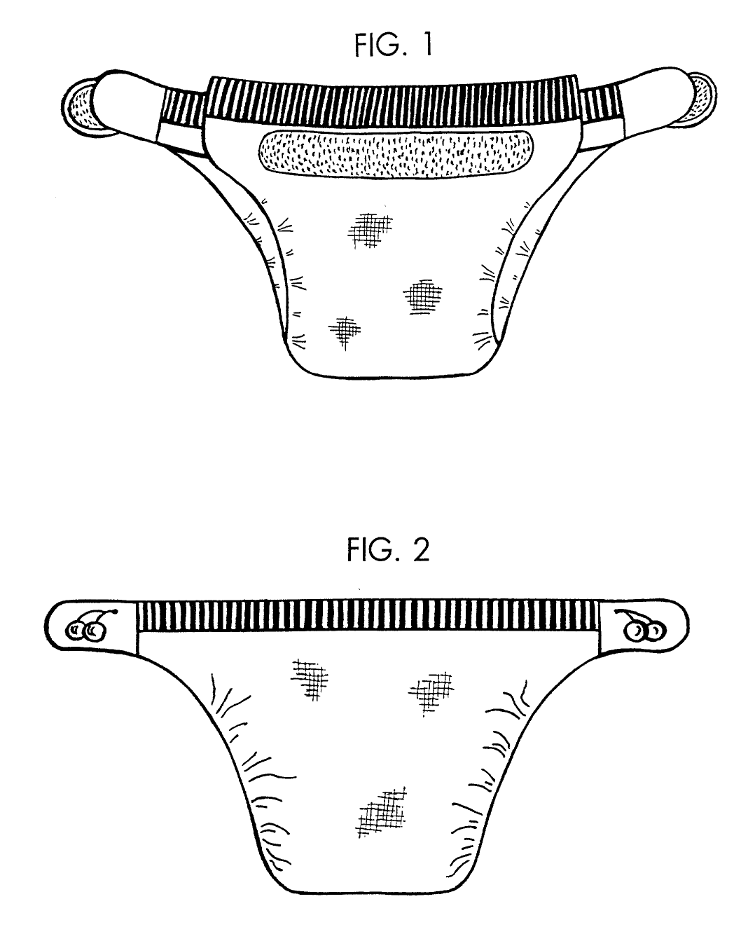

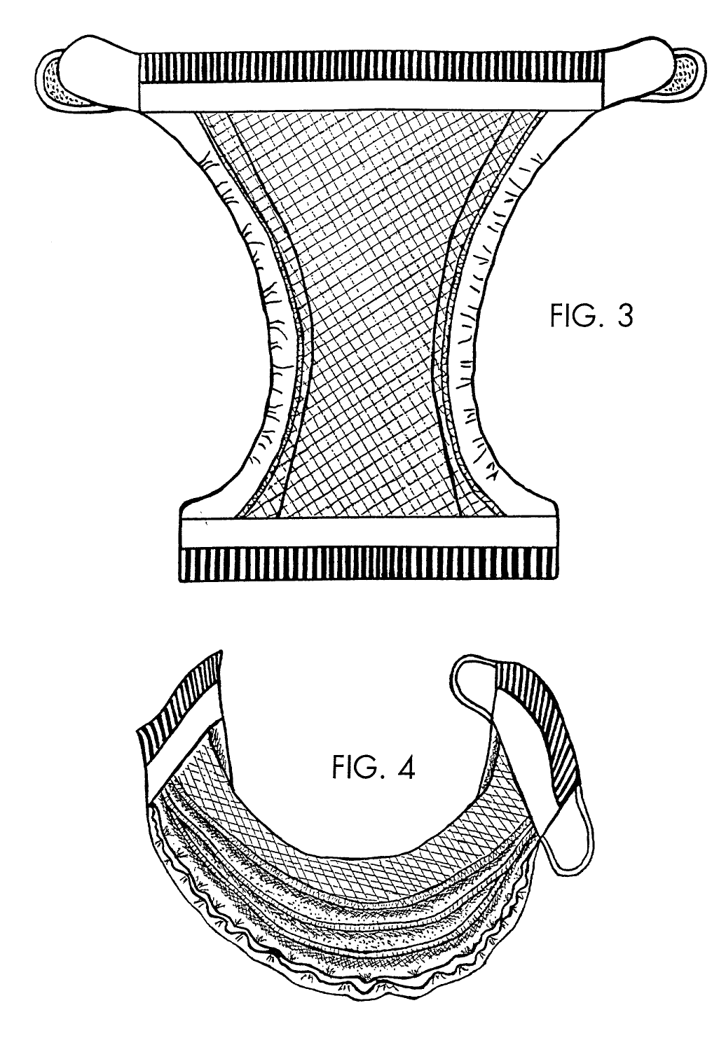

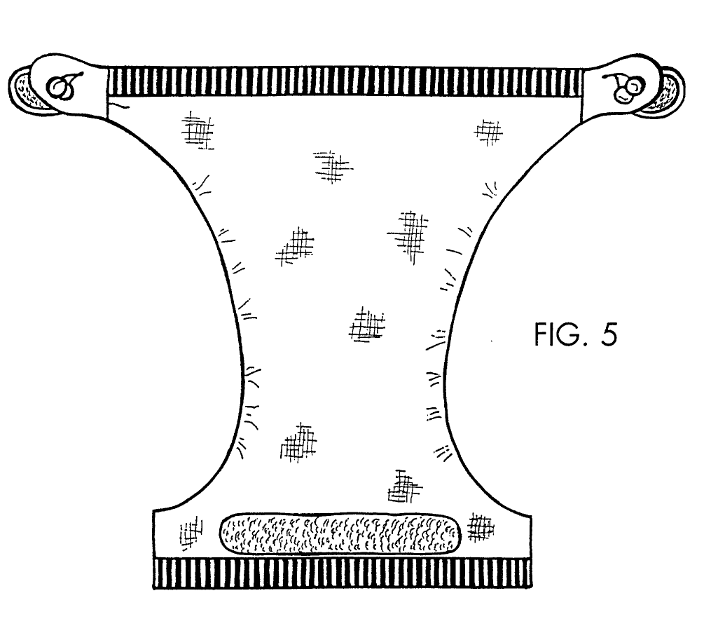

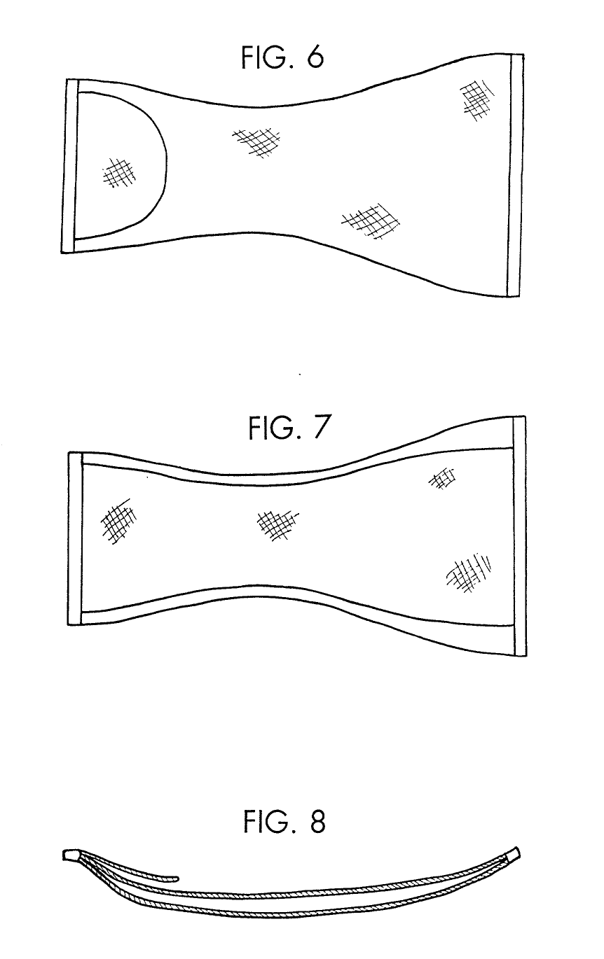

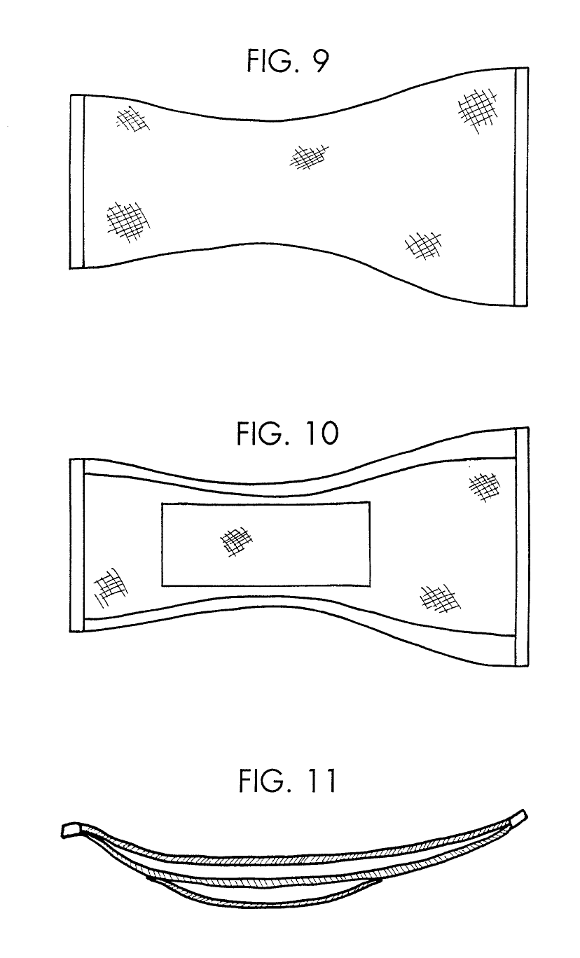

### DETAILED DESCRIPTION OF INVENTION

The invention, REUSABLE DIAPER WITH LINER, can be used for all children, birth to three years. It may also be used for adults with incontinence. Because it is an All-In-One diaper it is easy to use, and wash. It is different from existing diapers in many ways;

1. the nylon mesh holds the “poop” for easy elimination;

2. the nylon strips are guards to prevent “poop” going up the back;

3. the separate layers of flannel lining speed up the drying process;

4. the soft flexible fabric around waist gives comfort;

5. the double nylon outer shell prevents leaks;

6. the backing on the Velcro® fastener has a cover to prevent it from sticking to other fabrics;

7. different size and location, for boys and girls, for the third layer of the flannel piece was added to ensure extra absorption in those heavy wetting areas;

8. the edges around baby's legs are soft without making marks on baby's skin.

The diaper can be constructed with water-proof 100% nylon fabric, 100% cotton flannel fabric, 100% nylon mesh fabric, 65/35% polyester for fabric around waist, elastic, and Velcro® fastener, (applique if desired).

What I claim as my invention is:

1. The structural design for a REUSABLE DIAPER WITH LINER as shown and described.
